(19) United States
(12) Patent Application Publication
(10) Pub. No.: US 2011/0120478 A1
(43) Pub. Date: May 26, 2011

(54) BACKBOARD IMMOBILIZATION DEVICE

(75) Inventors: Jason A. Thompson, Springfield, PA (US); Dean Hobson, Aurora, CO (US)

(73) Assignee: Jake Systems LLC, Springfield, PA (US)

(21) Appl. No.: 12/933,746

(22) PCT Filed: Mar. 25, 2009
(86) PCT No.: PCT/US2009/038263

§ 371 Date: Feb. 3, 2011

Related U.S. Application Data
(60) Provisional application No. 61/039,216, filed on Mar. 25, 2008.

(51) Int. Cl. A61F 5/37

(52) U.S. Cl. 128/870

(57) ABSTRACT

A backboard immobilization device includes a first section having a plurality of interconnected panels and a second section having a like plurality of interconnected panels. A coupling assembly releasably couples the first section to the second section. The coupling assembly comprises a male insert having a plurality of ratchet teeth. The male insert is coupled to the first section. A female coupler has a pawl for releasable engagement with the plurality of ratchet teeth. The female coupler is coupled to the second section. An assembly including the backboard immobilization device and an inflatable air bladder assembly is also disclosed.

### CROSS-REFERENCE TO RELATED APPLICATION

The present application claims priority from U.S. Provisional Patent Application Ser. No. 61/039,216, which was filed on Mar. 25, 2008, and which is incorporated by reference herein in its entirety.

### FIELD OF THE INVENTION

The present invention relates to backboard immobilization devices. In particular, the present invention relates to backboard immobilization devices having complementary telescoping body support panels that may be releasably coupled together underneath a patient in order to stabilize and transport the patient.

### BACKGROUND OF THE INVENTION

Emergency medical personnel have found it to be desirable to immobilize a patient before removing the patient from the scene of an accident without having to lift, twist, turn, or roll the patient onto the immobilization device. Split stretchers have been found to be suitable for accomplishing such a task.

Conventional split stretchers, however, incorporate cumbersome locking mechanisms. Additionally, conventional split stretchers are typically provided having a predetermined length, which may be cumbersome in certain situations. Further, conventional stretchers are inappropriate for certain military uses, such as for special forces operations, which require compact, lightweight equipment.

### BRIEF SUMMARY OF THE INVENTION

Briefly, the present application provides a backboard immobilization device comprising a first section having a plurality of interconnected panels and a second section having a like plurality of interconnected panels. A coupling assembly releasably couples the first section to the second section. The coupling assembly comprises a male insert having a plurality of ratchet teeth and a female coupler having a pawl for releasable engagement with the plurality of ratchet teeth.

The present invention also provides a backboard immobilization assembly comprising a backboard including a first section, a second section, and a coupling assembly for releasably coupling the first section to the second section. The coupling assembly comprises a male insert having a plurality of ratchet teeth and a female coupler having a pawl for releasable engagement with the plurality of ratchet teeth. An air bladder assembly includes a first plurality of air bladders disposed within the first section and a second plurality of air bladders disposed within the second section. A pump is operatively coupled to and in fluid communication with the first and second plurality of bladders to pump a fluid into each of the first and second plurality of bladders.

### BRIEF DESCRIPTION OF THE DRAWINGS

The foregoing summary, as well as the following description of preferred embodiments of the invention, will be better understood when read in conjunction with the appending drawings, which are incorporated herein and constitute part of this specification. For the purposes of illustrating the invention, there are shown in the drawings exemplary embodiments of the present invention. It should be understood, however, that the invention is not limited to the precise arrangements and instrumentalities shown. In the drawing, the same reference numerals are employed for designating the same elements through out the several figures. In the drawings:

FIG. 1 is a perspective view of a backboard immobilization device according to an exemplary embodiment of the present invention;

FIG. 2 is a bottom plan view of the backboard immobilization device shown in FIG. 1;

FIG. 3 is a bottom plan view of the backboard immobilization device of FIG. 2, with separated sections;

FIG. 4 is a perspective view of the backboard immobilization device of FIG. 2, partially assembled;

FIG. 5 is a sectional view of a torso panel of the backboard immobilization device of FIG. 4;

FIG. 6 is an enlarged perspective view of a panel locking mechanism used to secure adjacent panels of the backboard immobilization device, taken along circle 6 of FIG. 1;

FIG. 7 is sectional view of the panel locking mechanism taken along lines 7-7 of FIG. 6;

FIG. 8 is a perspective view of a panel coupling mechanism for coupling each of the panels of the backboard immobilization device of 1 to each other;

FIG. 9 is an enlarged view of a coupling insert of the coupling mechanism of FIG. 8;

FIG. 10 is a schematic drawing of an optional air bladder system for use with the backboard immobilization device of FIGS. 1-9, with an air manifold configured to inflate a set of air bladders;

FIG. 11 is a schematic view of the air manifold illustrated in FIG. 10, with the air manifold configured to inflate a different set of bladders;

FIG. 12 is a top plan view of an immobilization device according to another exemplary embodiment of the present invention; and

FIG. 13 is a sectional view of the immobilization device of FIG. 12, taken along lines 13-13 of FIG. 12.

### DETAILED DESCRIPTION OF THE INVENTION

Certain terminology is used herein for convenience only and is not to be taken as a limitation on the present invention. The terminology includes the words specifically mentioned, derivatives thereof, and words of similar import. The following describes an exemplary embodiment of the invention. It should be understood based on this disclosure, however, that the invention is not limited by the exemplary embodiments of the invention.

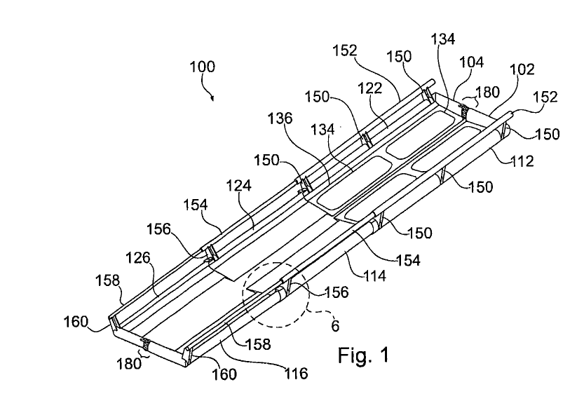

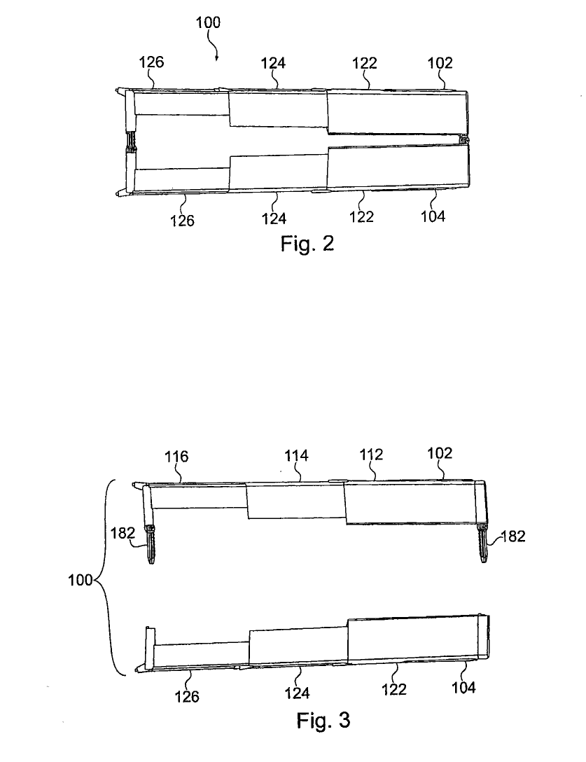

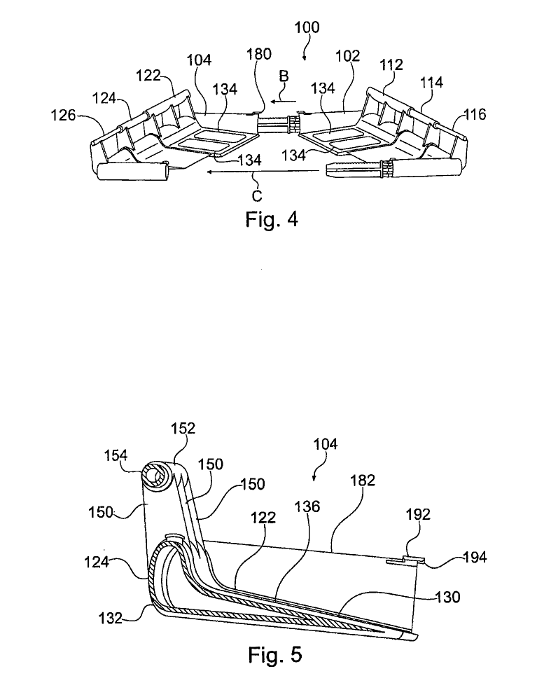

Referring to FIGS. 1 and 2, an exemplary embodiment of the present invention provides a backboard immobilization device 100 that is configured to be slid underneath a patient who is in a prone position. Immobilization device 100 is provided in a first section 102 and a second section 104 that are initially separated, as shown in FIG. 3. Each of first and second sections 102, 104 may be slid underneath the patient from a separate side of the patient and then connected together underneath the patient to form a unitary immobilization device 100. FIG. 4 illustrates the connection of first section 102 with second section 104. Once assembled, immobilization device 100, with the patient (not shown) lying on top, may then be transported to a location for further treatment.

Each of first and second sections 102, 104 includes a plurality of collapsible panels. Collapsible panels may include one or more possible arrangements of overlapping panels that slidably adjust relative to one another. Referring to FIGS. 1-4, first and second sections 102, 104 include telescoping panels. First section 102 includes a head panel 112, a torso panel 114, and a leg panel 116. Leg panel 116 is telescopically inserted within torso panel 114, with torso panel 114 being telescopically inserted within head panel 112. Similarly, second section 104 includes a head panel 122, a torso panel 124, and a leg panel 126. Leg panel 126 is telescopically inserted within torso panel 124, with torso panel being telescopically inserted within head panel 122. While three panels in each of first and second sections 102, 104 are shown, those skilled in the art will recognize that first and second sections 102, 104 may each incorporate more or less than three panels. Moreover, the sections need not be telescoping as shown, and instead may be interconnected by other overlapping arrangements.

Referring to FIG. 5, a sectional view of second section 104, taken through torso panel 124, is shown. Head panel 122 is a hollow member having a tapered inner portion 130 and a generally curved outer portion 132. Curved outer portion 132 also serves as a side protection to restrict movement of the patient while on immobilization device 100.

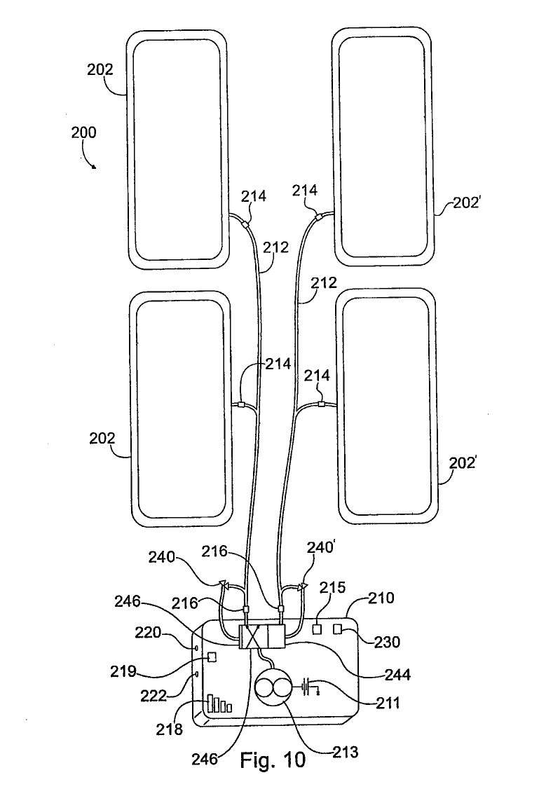

Referring back to FIGS. 1 and 4, each of head panels 112, 122 also includes a plurality depressed portions 134 on a top surface 136 of respective head panel 112, 122. Depressed portions 134 each accommodate an inflatable air bladder (shown in FIG. 10) that may be inflated and/or deflated to support the patient. While depressed portions 134 are shown only with respect to head portions 112, 122, those skilled in the art will recognize that depressed portions may also be formed in torso panels 114, 124 and leg panels 116, 126.

Referring now to FIGS. 1, 4, and 5, head panel 122 includes a plurality of generally vertical stanchions 150 that are spaced along the length of head panel 122 on curved outer portion 134. Stanchions 150 support a generally horizontal handle portion 152. Handle portion 152 is used to lift immobilization device 100. Handle portion 152 is hollow and, as shown in FIG. 5, telescopically receives handle portion 154. As shown in FIG. 1, handle portion 154 is coupled to torso panel 124 via a stanchion 156. Handle portion 154 is also hollow and telescopically receives a handle portion 158 that is coupled to leg panel 126 via a stanchion 160.

Stanchions 150, 156, 160 are sized and shaped to enable standard immobilization strap systems (not shown) to be removably attached thereto. Stanchions 150, 156, 160 may each include a single support, as shown in FIGS. 4 and 5, or alternatively, may each include a plurality of supports, as shown in FIG. 1. Immobilization strap systems include straps that are secured between each stanchion 150, 156, 160 and a corresponding stanchion 150, 156, 160 on first section 102.

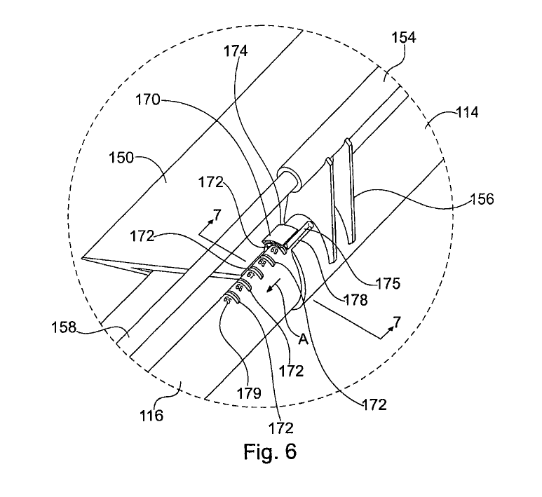

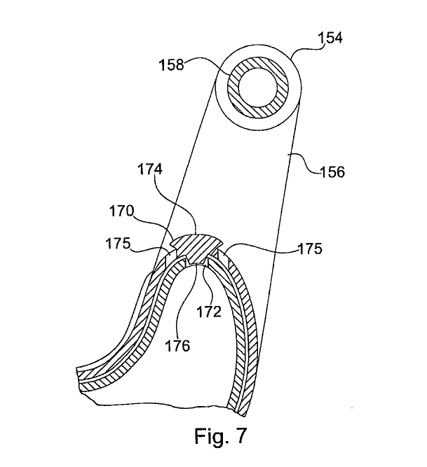

After panels 112, 114, 116, 122, 124, 126 are extended, they are locked in place relative to each other to restrict them from further extending or retracting on their own. As shown in FIGS. 6 and 7, leg panel 116 is shown extended from torso panel 114 by sliding leg panel 116 along arrow “A” with respect to torso panel 114. A locking mechanism 170 is used to lock leg panel 116 relative to torso panel 114.

Locking mechanism 170 includes a plurality of slots 172 formed in leg panel 116. A tab 174 extends from a free end of torso panel 114. Tab 174 is formed by cutting elongated slots 175 in torso panel 114. Tab 174 includes a tang 176 that extends into one of slots 172 and a lifting lip 178 that may be lifted to bias tang 176 out of slot 172 so that leg panel 116 may be extended or retracted relative to torso panel 114. When lifting lip 178 is released, tang 176 slides into slot 172, precluding movement of leg panel 116 with respect to torso panel 114. In an exemplary embodiment, adjacent slots 172 are spaced at one inch increments from each other along leg panel 116. While five slots 172 are shown, those skilled in the art will recognize that more or less than five slots 172 may be used. Indicia 179, such as numbers representing dimensions, will enable personnel using immobilization device 100 to extend leg panels 116, 126 an equivalent distance from their respective torso panels 114, 124.

Similarly, although not shown, locking mechanism 170 may also be used to restrict torso panel 114 from extending or retracting on its own relative to head panel 112. While locking mechanism 170 used with leg panel 116 and torso panel 114 includes a plurality of slots 172, in an exemplary embodiment, locking mechanism 170 used with torso panel 114 and head panel 112 uses only a single slot 172, so that torso panel 114 is extended a preset distance from head panel 112. With this arrangement, immobilization device 100 may be extended a distance sufficient to fully support a patient by adjusting the extension of torso panels 114, 124 from their respective leg panels 116, 126 and/or head panels 112, 122.

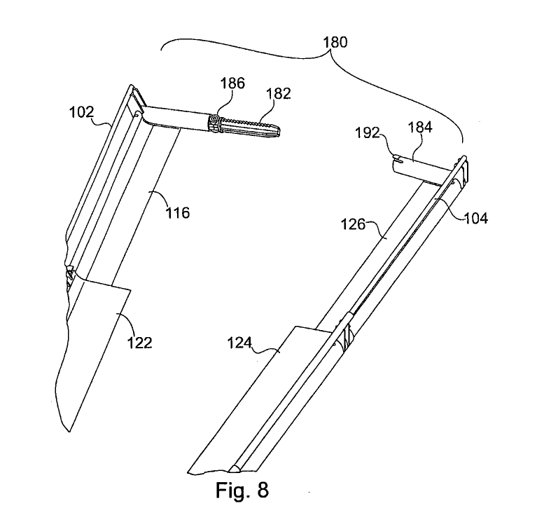

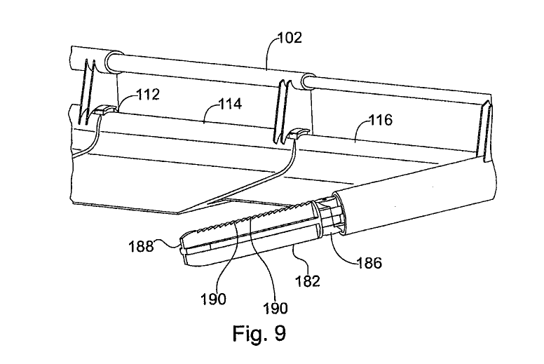

Referring now to FIGS. 8 and 9, a coupling assembly 180 that is used to couple first section 102 to second section 104 is shown. While coupling assembly 180 is shown in FIGS. 8 and 9 as associated with leg panels 116, 126, a second coupling assembly 180, shown in FIG. 1, is also associated with head panels 112, 122.

Coupling assembly 180 includes an elongated male insert 182 that is adapted to be inserted into and coupled with a female coupler 184 disposed on an end of leg panel 126 that is disposed away from torso panel 124. Insert 182 is hingedly coupled to coupling assembly via a hinge 186 having a vertical axis such that insert 182 pivots about hinge 186 along a horizontal plane generally parallel to the planar orientation of the panels. In an exemplary embodiment, insert 182 has a generally “cross-shaped” cross section, with a vertical prong 188 including a plurality of ratchet teeth 190.

Female coupler 184 includes a tab 192 that engages ratchet teeth 190 when insert 182 is inserted into female coupler 184. Tab 192 includes a pawl 194 (shown in FIG. 5) that rides over ratchet teeth 190 as insert 182 is inserted into female coupler 184. Tab 192 is ordinarily biased downwardly into engagement with ratchet teeth 190 to prevent insert 182 from being withdrawn from female coupler 184. Tab 192 may be flexed upwardly to remove pawl 194 from ratchet teeth 190 and allow insert 182 to be withdrawn from coupler 184.

While inserts 182 are shown in the Figures (particularly FIG. 3) as being fixed to first section 102, those skilled in the art will recognize that one or both inserts 182 may alternatively be fixed to second section 104.

Referring now to FIG. 10, an inflatable bladder assembly 200 is shown. Bladder assembly 200 includes a first set of bladders 202 that may be releasably inserted into depressed portions 134 in head portion 112 and a second set of bladders 202′ that are releasably inserted into depressed portions 134 in head portion 122. Bladder assembly 200 is used to systematically raise and lower different parts of the patient's body relative to immobilization device 100 to simulate the rhythm of sleep and to reduce necrosis of the patient as a result of immobility resulting from patient's injury and/or securement to immobilization device 100. While two bladders are illustrated with each set of bladders 202, 202′, those skilled in the art will recognize that more or less than two bladders may be used with each set of bladders 202, 202′. By way of example only, three bladders may be used, with a bladder located under the patient's shoulder blade, a bladder located under the patient's buttocks, and a bladder located under the patient's calf/ankle.

Each set of bladders 202, 202′ is in fluid communication with assembly 210 via a dedicated fluid supply line, such as, for example, an air supply line 212. A coupling 214 proximate to each set of bladders 202, 202′ releasably couples each set of bladders 202, 202′ to its respective air supply line 212, while a coupling 216 proximate to pump assembly 210 releasably couples each air supply line 212 to pump assembly 210.

In an exemplary embodiment, pump assembly 210 includes its own power source, represented by a battery 211 in FIG. 10. A battery strength indicator 218 and a “Low Battery Power” audio/visual alarm 219 provide a user with an indication of battery condition. An AC connection 220 and a DC connection 222 provide for external power connections to pump assembly 210.

Pump assembly 210 includes a pump 213 that sequentially pumps air into each set of bladders 202, 202′ to sequentially lift and lower patient relative to immobilization device 100. Pump assembly 210 also includes a sensor 215 that senses an obstruction in any air supply line 212 to a set of bladders 202, 202′, and transmits a signal to an audio/visual alarm 230 to alert the user to the obstruction.

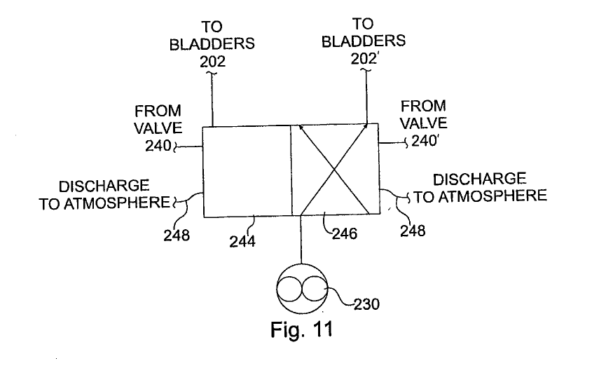

Each set of bladders 202, 202′ is in fluid communication with a relief valve 240, 240′, respectively, that releases air from within its respective set of bladders 202, 202′ upon air pressure within the bladder exceeding a predetermined value. Relief valves 240, 240′ are coupled to a manifold 244 that is fluidly located between pump 213 and bladders 202, 202′. Manifold 244 may include a sliding valve 246 that operates between the position in FIG. 10, which directs air from pump 213 to bladders 202, and the position in FIG. 11, which directs air from pump 213 to bladders 202′. Sliding valve 246 may be air actuated, such as by air released from valves 240, 240′. Alternatively, sliding valve 246 may be electrically actuated in response to an electrical signal generated at valves 240, 240′ when valves 240, 240′ are releasing air from their respective sets of bladders 202, 202′. If sliding valve 246 incorporates air that is released from the sets of bladders 202, 202′, manifold 244 may also incorporate exhaust ports 248 (shown in FIG. 11), that exhaust the air from manifold 244.

The components of immobilization device 100 may be constructed from a non-metallic radiograph transparent material such as, for example, carbon fiber or Kevlar®. Alternatively, immobilization device 100 may be constructed from other materials.

In a compressed state, with torso panel 114 fully inserted into head panel 112 and with leg panel 116 fully inserted into torso panel 114, first section 102 of immobilization device 100 has an exemplary length of between about 96.5 cm (about 38 inches) and about 106.7 cm (about 42 inches) and a preferred width of between about 20.3 cm (about 8 inches) and about 22.9 cm (about 9 inches). In a fully extended and assembled state, immobilization device 100 has a preferred length of between about 165.1 cm (about 65 inches) and about 213.4 cm (about 84 inches) and a preferred width of between about 38.1 cm (about 15 inches) and about 45.7 cm (about 18 inches). Excluding bladder assembly 200, immobilization device 100 preferably weighs between about 4.1 kilograms (about 9 pounds) and about 7.3 kilograms (about 16 pounds).

Immobilization device 100 may be stored with first section 102 separate from second section 104. First and second sections 102, 104 may be stored in a compressed state, with leg panels 116, 126 disposed within their respective torso panels 114, 124, and with torso panels 114, 124 disposed within their respective head panels 112, 122.

In use, first section 102 is disposed on one side of the patient, and second section 104 is disposed on the opposite side of the patient. Torso panels 114, 124 are fully extended from there respective head panels 112, 122. Leg panels 116, 126 are next extended from their respective torso panels 114, 124 a distance commensurate with the height of the patient. In an exemplary embodiment, leg panels 116, 126 are extended only far enough from their respective torso panels 114, 124 to enable the patient to be fully contained within immobilization device 100.

Head panels 112, 122 are first coupled together by engaging coupling assembly 180 in a direction shown by arrow “B” in FIG. 4. Pawl 194 has a tapered projection that rides over ratchet teeth 190 to allow insert 182 to be slid into female coupling 184 without having to lift tab 192.

With patient being scooped by immobilization device 100, leg panels 116, 126 are then coupled together by engaging coupling assembly 180 in a direction shown by arrow “C” in FIG. 4. Hinges 186 on each of coupling assemblies 180 allow first section 102 to pivot with respect to second section 104 after head panels 112, 122 are coupled together, and to allow leg panels 116, 126 to be coupled together. That is, inserts 182 are each pivotable with respect to their associated panels so as to align with the corresponding female coupler 184 during connection of first and second sections 102, 104.

After the patient is placed on immobilization device 100, the patient will be strapped to immobilization device 100 using any of a number of commercially available immobilization strap systems (not shown). Air supply lines 212 from each set of bladders 202, 202′ are connected to pump assembly 210 via couplings 216. Pump assembly 210 may be started to alternately inflate each of bladders 202, 202′ on head panels 112, 122. Pump assembly 210 may be placed on immobilization device 100 proximate to the patient's feet for transport.

An exemplary operation of air bladder assembly 200 will now be described. Pump 213 pumps air into manifold 244. When sliding valve 246 is positioned within manifold 244 in the position illustrated in FIG. 10, air from pump 213 flows through manifold 244 to inflate set of bladders 202. Bladders 202 lift one side of the patient upward from immobilization device 100.

When the pressure within set of bladders 202 exceeds a predetermined value, excess air pressure is relieved from bladders 202 via relief valve 240, which directs air to slide valve 246, moving sliding valve 246 from the position shown in FIG. 10 to the position shown in FIG. 11. Relief of the air pressure from set of bladders 202 also lowers the patient onto immobilization device 100.

In the position shown in FIG. 11, air from pump 213 flows through manifold 244 and inflates set of bladders 202′, lifting the other side of the patient from immobilization device 100. When the pressure within set of bladders 202′ exceeds a predetermined value, excess air pressure is relieved from bladders 202′ via relief valve 240′, which directs air to slide valve 246, moving sliding valve 246 from the position seen in FIG. 11 back to the position seen in FIG. 10, and the above-described process is repeated as long as pump 213 operates. Relief of the air pressure from set of bladders 202′ also lowers the patient onto immobilization device 100.

After use, immobilization device 100 may be disassembled for storage by removing bladder assembly 200 from immobilization device 100. First section 102 may be separated from second section 104 by lifting tab 192 and withdrawing insert 182 from female coupling 184 at each of head panels 112, 122 and leg panels 116, 126.

Leg panels 116, 126 may then be retracted into their respective torso panels 114, 124 by lifting insert 176 from slot 172 and pushing leg panels 116, 126 into their respective torso panels 114, 124. Similarly, torso panels 114, 124 may be retracted into their respective head panels 112, 122 by lifting insert 176 from slot 172 and pushing torso panels 114, 124 into their respective head panels 112, 122.

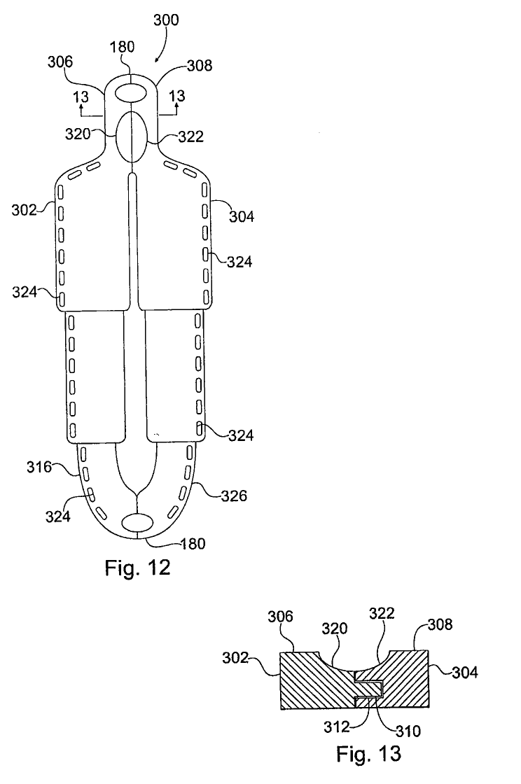

An alternative embodiment of an immobilization device 300 according to an aspect of the present invention is illustrated in FIGS. 12 and 13. Immobilization device 300 includes first and second sections 302, 304, respectively. First and second sections 302, 304 include extensions 306, 308 that mate to accommodate the patient's head. Extension 306 include a tongue 310 that is inserted into a groove 312 in extension 308. This tongue-and-groove mating arrangement provides additional strength to immobilization device 300. Similarly, a tongue-and-groove mating arrangement may be incorporated into leg panels 316, 326, as well.

Coupling assembly 180 (illustrated in FIGS. 4, 8, and 9) may be used to releasably secure first and second sections 302, 304 to each other. Each of extension 306, 308 may include a cutout portion 320, 322, respectively, that forms a depression for receiving the patient's head when first and second sections 302, 304 are coupled to each other.

Optionally, instead of the stanchions 150, 152, 160 described above that are used to support handle portions 152, 154, 158, handhold cutouts 324 may be formed around the outer perimeter of each of first and second sections 302, 304 to enable medical/rescue personnel to grip and lift immobilization device 300.

Although not shown in FIGS. 12 and 13, air bladder assembly 200 may be used with immobilization device 300.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A backboard immobilization device comprising:
a first section having a plurality of first interconnected panels;
a second section having a like plurality of second interconnected panels;
a coupling assembly for releasably coupling the first section to the second section, the coupling assembly comprising:
a male insert having a plurality of ratchet teeth, the male insert being coupled to the first section; and
a female coupler having a pawl for releasable engagement with the plurality of ratchet teeth, the female coupler being coupled to the second section.

2. The backboard immobilization device according to claim 1, wherein at least one of the plurality of interconnected panels comprises top surface, the top surface having a plurality of depressed portions formed therein.

3. The backboard immobilization device according to claim 2, further comprising an air bladder inserted into each of the depressed portions.

4. The backboard immobilization device according to claim 3, further comprising a pump in fluid communication with each of the air bladders.

5. The backboard immobilization device according to claim 1, further comprising a first plurality of air bladders disposed on the first section and a second plurality of air bladders disposed on the second section.

6. The backboard immobilization device according to claim 5, further comprising a pump in fluid communication with each of the first and second plurality of air bladders.

7. The backboard immobilization device according to claim 6, wherein the pump alternates fluid communication between the first plurality of air bladders and the second plurality of air bladders.

8. The backboard immobilization device according to claim 1, wherein the male insert is hingedly coupled to the first section.

9. The backboard immobilization device according to claim 1, wherein the first interconnected panels comprise telescoping panels.

10. The backboard immobilization device according to claim 1, wherein each of the plurality of interconnected panels comprises a tapered inner portion and a generally curved outer portion.

11. A backboard immobilization assembly comprising:
a backboard including:
a first section;
a second section;
a coupling assembly for releasably coupling the first section to the second section, the coupling assembly comprising:
a male insert having a plurality of ratchet teeth; and
a female coupler having a pawl for releasable engagement with the plurality of ratchet teeth; and
an air bladder assembly including:
a first plurality of air bladders disposed within the first section;
a second plurality of air bladders disposed within the second section;
a pump operatively coupled to and in fluid communication with the first and second plurality of bladders to pump a fluid into each of the first and second plurality of bladders.

12. The backboard immobilization assembly according to claim 11, wherein the pump is operable to sequentially pump a fluid into each of the first and second plurality of bladders.

13. The backboard immobilization assembly according to claim 11, wherein each of the first and second plurality of air bladders each includes a separate supply line in fluid communication with the pump.

14. The backboard immobilization assembly according to claim 12, wherein the pump alternates fluid communication between the first plurality of air bladders and the second plurality of air bladders.
